(19) United States
(12) Patent Application Publication
(10) Pub. No.: US 2018/0146954 A1
(43) Pub. Date: May 31, 2018

(54) METHOD OF ULTRASOUND APPARATUS PARAMETERS CONFIGURATION AND AN ULTRASOUND APPARATUS OF USING THE SAME

(71) Applicant: General Electric Company, Schenectady, NY (US)

(72) Inventors: Jiajiu Yang, Wuxi (CN); Zhiqiang Jiang, Wuxi (CN); Ke Tao, Wuxi (CN)

(21) Appl. No.: 15/827,543

(22) Filed: Nov. 30, 2017

(30) Foreign Application Priority Data
Nov. 30, 2016 (CN) 201611082165.3

(51) Int. Cl. A61B 8/08; A61B 8/00

(52) U.S. Cl. CPC A61B 8/5246; A61B 8/463; A61B 8/465; A61B 8/4472; A61B 8/0833; A61B 8/4461; A61B 8/5223; A61B 8/5207

(57) ABSTRACT

A method and ultrasound apparatus for configuring a parameter of the ultrasound apparatus includes pre-obtaining at least two corresponding ultrasound images according to at least two pre-set values of the parameter. The method includes displaying the at least two ultrasound images simultaneously, selecting one of the displayed ultrasound images, and configuring the parameter to a pre-set value corresponding to the selected ultrasound image.

### TECHNICAL FIELD

The present invention relates to a method for configuring a parameter of an ultrasound apparatus and an ultrasound apparatus using the method.

### BACKGROUND

At present, as technology advances, ultrasound apparatuses are becoming smaller and easier to use with more powerful functions. Ultrasound imaging has been used in various fields in hospitals, such as radiology, cardiac surgery, emergency treatment, obstetrics, gynaecology and surgical operations. Physicians in all fields have begun to use ultrasound apparatus as one of diagnostic tools in their daily work.

However, parameters of ultrasound apparatuses need to be set when the ultrasound scan examination is performed, and the values of these parameters are determined based on many complex factors. For example, it is necessary to adjust the frame frequency, focus position, frequency, depth, number of focuses, frame average, greyscale map, pseudo-colour, suppression, lateral smoothing and other parameters. Therefore, it is required that physicians who perform the scan have a higher technical level and rich clinical experience to determine appropriate parameter values.

However, due to the increasing of ultrasound imaging technology in application fields, physicians who need to use ultrasound imaging may come from various fields, such as departments of cardiac surgery, obstetrics and gynaecology, surgery and inpatient; and moreover, some of these physicians are novices, and they may be unfamiliar with the ultrasound imaging technology and also lack corresponding operational knowledge and experience. Consequently, they may not get a clear scan image when using an ultrasound scanning apparatus in clinical work, which affects the effect of ultrasound imaging examination.

Therefore, it is necessary to propose a new method for configuring a parameter of an ultrasound apparatus and a new ultrasound apparatus.

### BRIEF DESCRIPTION OF THE INVENTION

The above-mentioned shortcomings, disadvantages, and problems are addressed herein which will be understood by reading and understanding the following specification.

In an embodiment, a method for configuring a parameter of an ultrasound apparatus includes pre-obtaining at least two corresponding ultrasound images according to at least two pre-set values of the parameter. The method includes displaying the at least two ultrasound images simultaneously, selecting one of the displayed ultrasound images, and configuring the parameter to a pre-set value corresponding to the selected ultrasound image.

In an embodiment, an ultrasound apparatus includes a processing unit configured to obtain at least two corresponding ultrasound images according to at least two pre-set values of a parameter. The ultrasound apparatus includes a display unit for displaying the at least two ultrasound images simultaneously. The processing unit is configured to receive a command of selecting one of the ultrasound images and to configure the parameter to a pre-set value corresponding to the selected ultrasound image.

Various other features, objects, and advantages of the invention will be made apparent to those skilled in the art from the accompanying drawings and detailed description thereof.

### BRIEF DESCRIPTION OF THE DRAWINGS

The present invention will be better understood by means of describing exemplary embodiments of the present invention in conjunction with the accompanying drawings, in which:

FIG. 1 shows a module diagram according to an embodiment of an ultrasound apparatus of the present invention;

FIG. 2 is a flow chart of a method for configuring a parameter of an ultrasound apparatus provided in the present invention;

FIG. 3 shows a schematic diagram of displaying of a display unit according to an embodiment of the method for configuring a parameter of an ultrasound apparatus of the present invention;

FIG. 4 shows a greyscale mapping curve according to an embodiment of the method for configuring a parameter of an ultrasound apparatus of the present invention;

FIG. 5 shows a schematic diagram of displaying of a display unit according to another embodiment of the method for configuring a parameter of an ultrasound apparatus of the present invention; and

FIG. 6 shows a schematic diagram of displaying of a display unit according to another embodiment of the method for configuring a parameter of an ultrasound apparatus of the present invention.

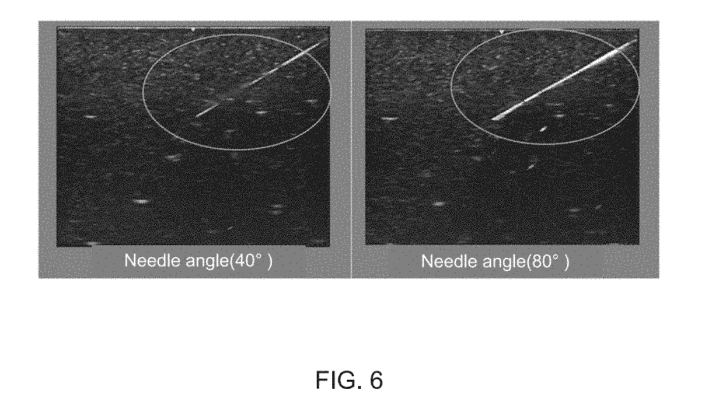

### DETAILED DESCRIPTION OF THE DRAWINGS

Hereafter, a detailed description will be given for preferred embodiments of the present disclosure. It should be pointed out that in the detailed description of the embodiments, for simplicity and conciseness, it is impossible for the Description to describe all the features of the practical embodiments in details. It should be understood that in the process of a practical implementation of any embodiment, just as in the process of an engineering project or a designing project, in order to achieve a specific goal of the developer and in order to satisfy some system-related or business-related constraints, a variety of decisions will usually be made, which will also be varied from one embodiment to another. In addition, it can also be understood that although the effort made in such developing process may be complex and time-consuming, some variations such as design, manufacture and production on the basis of the technical contents disclosed in the disclosure are just customary technical means in the art for one of ordinary skilled in the art associated with the contents disclosed in the present disclosure, which should not be regarded as insufficient disclosure of the present disclosure.

Unless defined otherwise, all the technical or scientific terms used in the Claims and the Description should have the same meanings as commonly understood by one of ordinary skilled in the art to which the present disclosure belongs. The terms “first”, “second” and the like in the Description and the Claims of the present utility model do not mean any sequential order, number or importance, but are only used for distinguishing different components. The terms “a”, “an” and the like do not denote a limitation of quantity, but denote the existence of at least one. The terms “comprises”, “comprising”, “includes”, “including” and the like mean that the element or object in front of the “comprises”, “comprising”, “includes” and “including” covers the elements or objects and their equivalents illustrated following the “comprises”, “comprising”, “includes” and “including”, but do not exclude other elements or objects. The term “coupled” or “connected” or the like is not limited to being connected physically or mechanically, nor limited to being connected directly or indirectly.

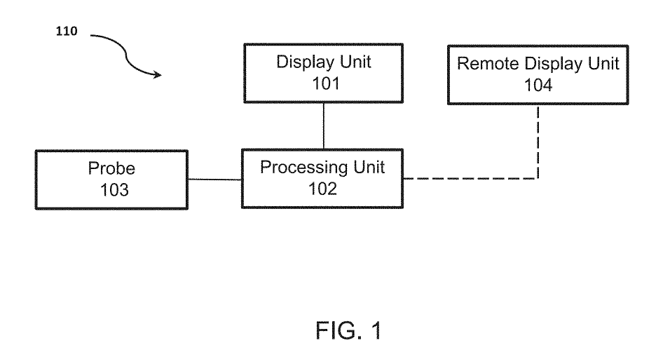

FIG. 1 shows a module diagram according to an embodiment of an ultrasound apparatus of the present invention. An ultrasound apparatus 10 comprises a display unit 101, a processing unit 102 and a probe 103. The processing unit 102 comprises control and processing devices, such as a digital signal processor (DSP), a CPU and a field programmable gate array (FPGA). The processing unit 102 controls the probe 103 (including all known types of probes) to emit a pulse signal to a detection object, the probe 103 receives a reflected reflection pulse and transmits same to the processing unit 102, and the processing unit 102 processes the reflection pulse and obtains an ultrasound image. Next, the processing unit 102 displays the ultrasound image on the display unit 101 (including all known types of display screens and display devices). In addition, input/output interaction interfaces (including a touch screen or physical buttons) of the entire apparatus are all controlled by the processing unit 102.

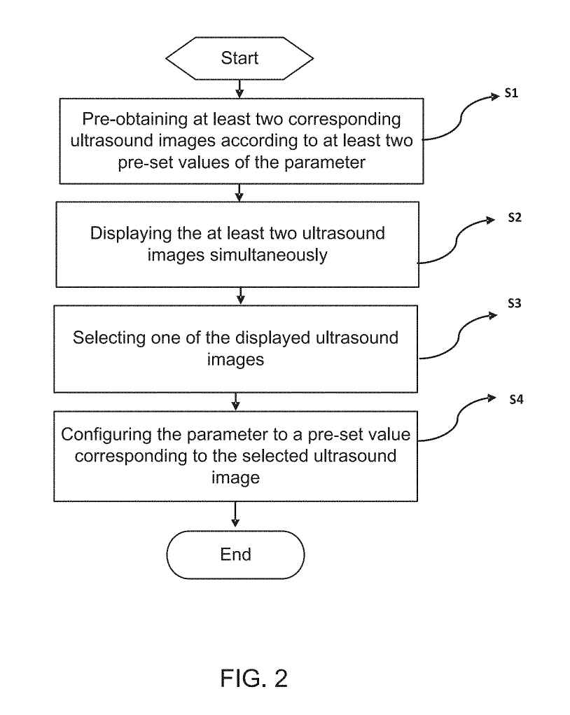

FIG. 2 provides a flow chart of a method for configuring a parameter of an ultrasound apparatus provided in the present invention. As soon as a physician turns on the ultrasound apparatus 10 and then directs the probe 103 towards the detection object, the display unit 101 of the ultrasound apparatus 10 will display an ultrasound image, and the ultrasound image at this moment is obtained according to a boot default value of each parameter. Once the physician inputs a command to configure a certain parameter through the interaction interface, the processing unit 102 of the ultrasound apparatus will receive the parameter configuration command.

First, proceeding to step S1, the processing unit 102 of the ultrasound apparatus 10 invokes at least two predefined values for a parameter needing to be configured, and respectively processes, when the parameter is set with different values, the values to obtain at least two corresponding ultrasound images.

Further, proceeding to step S2, the display unit 101 of the ultrasound apparatus 10 displays the obtained at least two ultrasound images simultaneously, and the physician can see, on the display unit 101, the effect of the ultrasound images with setting the different values of the parameter.

Subsequently, proceeding to step S3, the physician selects a satisfactory one from the at least two ultrasound images and inputs a command for selecting the ultrasound image through the interaction interface.

Next, proceeding to step S4, the processing unit 102 of the ultrasound apparatus 10 configures, according to the ultrasound image selected by the physician, the parameter to a pre-set value corresponding to the image. In the following period of time, the parameter is configured by the processing unit 102 to the pre-set value for processing and displaying the ultrasound image.

Before proceeding to step S1, the processing unit 102 of the ultrasound apparatus 10 controls the probe 103 to emit an emission pulse to a detection object. In practical applications, parameters that need to be configured for the ultrasound apparatus include pre-processing parameters and post-processing parameters. The pre-processing parameters include the frame frequency, focus position, frequency, depth, number of focuses, energy output, virtual convex array, packet size, etc. The post-processing parameters include the frame average, greyscale map, pseudo-colour, suppression, lateral smoothing, etc.

In some application scenarios, parameters needing to be configured belong to the pre-processing parameters. The emission pulses emitted by the probe 103 under the control of the processing unit 102 are thus different from each other, and there is a one-to-one correspondence between the emission pulses and the pre-set values. The probe 103 collects reflected reflection signals (there is also a one-to-one correspondence between the reflected signals and the pre-set values). The processing unit 102 processes the reflection signals respectively to obtain at least two corresponding ultrasound images.

In certain application scenarios, parameters needing to be configured belong to the post-processing parameters, that is, the configuration of image-related parameters. As a result, parameters of a signal link are invariable, and for different pre-set values, the emission pulse does not need to be changed. Therefore, it is possible that only one emission pulse can be emitted. The probe 103 collects the reflection signal reflected by the emission pulse. During image processing, the processing unit 102 processes the reflection signal according to different pre-set values of the parameter so as to obtain at least two ultrasound images. Further, in some physician teaching training applications, the reflection signal may not be obtained from a pulse emitted in real time, but may be pre-stored in the ultrasound apparatus. During teaching and training, it is not necessary to control the probe to emit the pulse, but necessary to process a certain post-processing parameter of the stored reflection pulse respectively. In a similar way, multiple processed images are obtained and displayed in the same interface.

The pre-set values in the present invention are generally empirical values in the prior art and are pre-stored within the ultrasound apparatus. In some cases, the adjusted parameter is a continuous value, for example, from 0 to 100, and the pre-set values may be pre-customized according to the physician's input, such as 0, 50 and 100 or 0, 20, 60 and 100, etc. The pre-set values may also be continuously updated by taking the value selected by a physician each time as one of the pre-set alternative values next time. A similar method would have been easily conceivable to those skilled in the art, and therefore the details will not be repeated here.

As shown in FIG. 1, the ultrasound apparatus 10 further comprises a remote display unit 104. The remote display unit 104 performs displaying synchronously with the local display unit 101 of the ultrasound apparatus in a wired or wireless manner. In certain telemedicine application scenarios, for novice physicians in remote areas, when configuring a certain parameter, the novice physicians operate locally and experienced physicians operate remotely. The remote physician can select one from at least two displayed ultrasound images, and the processing unit 102 thus configures the parameter according to a pre-set value corresponding to the ultrasound image. Novice physicians can operate according to the configured parameters.

In certain application situations, for example, where the detection object is heart, when a certain parameter for displaying the heart needs to be adjusted, the ultrasound image is substantially a dynamically displayed ultrasound image frame, i.e., a segment of video. The same method may also be used, i.e., the display unit simultaneously displays processed dynamic ultrasound image frames, and after a certain segment of image frame is selected, the corresponding parameter may also be set to a pre-set value corresponding to the ultrasound image frame.

In order to deepen the understanding of a skilled person to the present invention, the present invention will be further described hereinafter by way of several embodiments in conjunction with the accompanying drawings.

### Embodiment I

In a certain application situation, a physician needs to adjust a parameter, i.e. a measurement depth of the ultrasound apparatus 10, such that the display range reaches the depth at which the region of interest (ROI) is located. In an existing ultrasound apparatus, the physician adjusts the depth parameter by means of a knob and adjusts same step by step to a satisfactory value, which needs to take a considerable amount of time.

In this embodiment, after the physician clicks the button for adjusting the depth (in other embodiments, it is also possible to click on a button on a touch screen), the ultrasound apparatus 10 receives the command, and the ultrasound apparatus 10 invokes four pre-set values in a cache (which may be other storage devices in other embodiments, including but not limited to a floppy disk, a hard disk, an optical disk, an SD card, an internal memory, etc.). In this embodiment, the pre-set values are 9 cm, 14 cm, 18 cm and 22 cm. The four pre-set values here are empirical values widely used in the prior art. There may be more than just four pre-set values, and may also be more options.

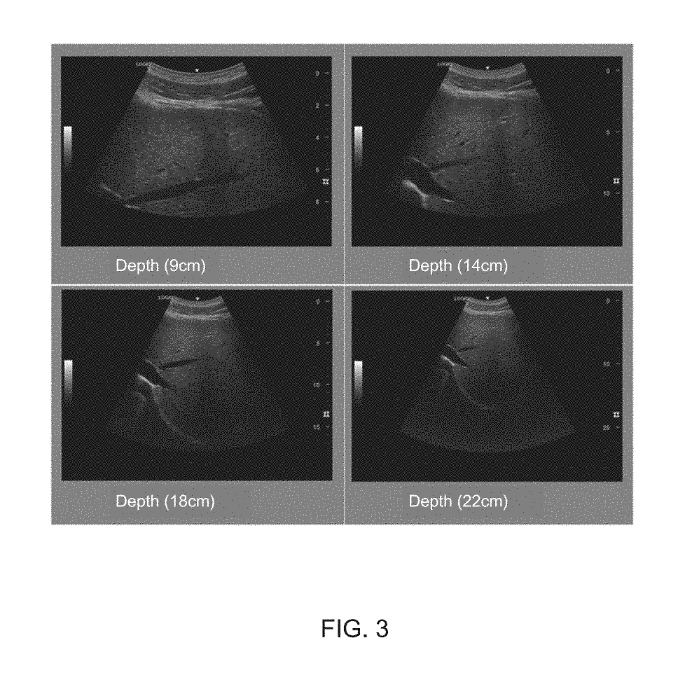

The depth of the ultrasound signal is determined by the frequency of the ultrasound emission pulse. The processing unit 102 of the ultrasound apparatus 10 controls the ultrasound probe 103 to emit 4 emission pulses, respectively being ultrasound emission pulses of 8 megahertz (MHz), 6 megahertz (MHz), 4 megahertz (MHz) and 2 megahertz (the values here are only for the purpose of illustrating the principle, but are not real data). The probe 103 collects the reflected reflection signal, and four ultrasound images are obtained after waveform synthesis by the processing unit 102. The four ultrasound images are simultaneously displayed on the display unit 101 of the ultrasound apparatus 10, as shown in FIG. 3, which is a display schematic diagram of the display unit 101. In other embodiments, it is totally possible to display nine images or sixteen images on the display unit at the same time, depending on how many pre-set values there are. Now, the physician sees that the region of interest (ROI) displayed at the 18 cm configuration is the most complete and has the largest area within the display range, and clicks to select that image corresponding to 18 cm. Thus, the processing unit 102 performs configuration according to the depth of 18 cm and emits an emission pulse of 4 megahertz (MHz).

### Embodiment II

In an application situation of B-mode ultrasound, the ultrasound signal is presented on the display unit 101 in the form of a greyscale value of pixels. For example, for a greyscale value with a 8-bit depth, a greyscale interval range is from 0 to 255. In order to better display the region of interest (ROI) in contrast, it is necessary to call a greyscale mapping algorithm, which belongs to an image processing-related parameter. The principle of the greyscale mapping algorithm is that each greyscale value in the region is mapped to obtain another greyscale value by means of a mapping curve, such that a greyscale value of a desired region of interest (ROI) is enhanced while a greyscale value of a region of no interest is suppressed, thereby improving the display effect by changing the contrast between the greyscale values.

When using such an apparatus, a physician needs to adjust the parameter, i.e. greyscale mapping, that is, to determine which greyscale mapping curve is needed. In an existing ultrasound apparatus, the physician is required to select, in a targeted manner, a greyscale mapping curve when practically using the parameter, depending on whether a detected object is the artery or the liver, until a satisfactory display effect is achieved.

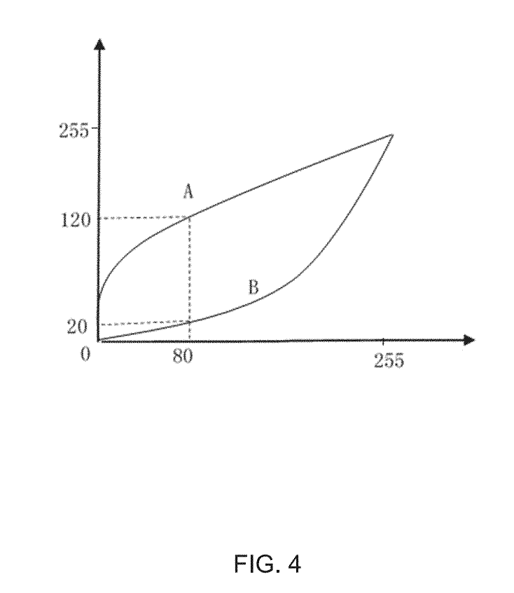

In this embodiment, after the physician clicks on the button for adjusting the greyscale mapping (in other embodiments, it is also possible to click on a button on a touch screen), the ultrasound apparatus 10 receives the command, and the ultrasound apparatus 10 invokes two greyscale mapping relationships in a cache (which may be other storage devices in other embodiments, including but not limited to a floppy disk, a hard disk, an optical disk, an SD card, an internal memory, etc.). Greyscale mapping relationship A and greyscale mapping relationship B are as shown in FIG. 4. In order to facilitate the illustration, the greyscale mapping curve A and the greyscale mapping curve B are drawn in the same coordinate system. The two greyscale mapping curves here are empirical values widely used in the prior art. There may be two pre-set greyscale mapping curves, or more options may also be provided.

The processing unit 102 of the ultrasound apparatus 10 controls the probe 103 to emit an emission pulse. In this embodiment, the greyscale mapping belongs to the post-processing parameter. The probe 103 only needs to emit one emission pulse, and the following post-processing parameters are all processed with regard to a reflection signal of this emission pulse. It is noteworthy that, in certain apparatuses, the probe 103 can emit at least two identical emission pulses. Since the detection object is substantially unchanged during an emission gap, a reflection signal obtained by emitting at least two identical emission pulses is the same as that obtained by emitting one emission pulse. It is therefore necessary to emphasize that the effect achieved by emitting at least two identical emission pulses is the same as that achieved by emitting one emission pulse, so that the two emission methods should be construed as one equivalent technical solution.

In the process of greyscale mapping, the processing unit 102 processes the reflection signal to obtain an original ultrasound image, and greyscale values of all the points of the original ultrasound image (including the region of interest and the region of no interest) are respectively mapped by using the greyscale mapping curve A and the greyscale mapping curve B. For example, a greyscale value of a certain point within the region of interest (ROI) is 80, and is mapped to 120 by means of the greyscale mapping curve A and is mapped to 20 by means of the greyscale mapping curve B, while a greyscale value of a certain point within the region of no interest is 140, and is mapped to 160 by means of the greyscale mapping curve A and is mapped to 150 by means of the greyscale mapping curve B. For each point, one-to-one mapping is performed by means of the greyscale mapping curves A and B respectively, and finally two ultrasound images are obtained.

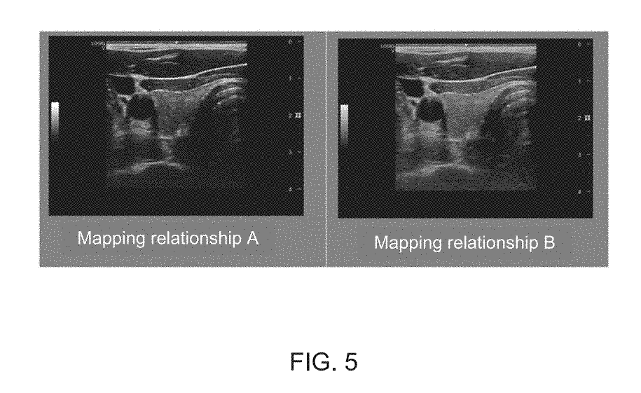

The two ultrasound images are simultaneously displayed on the display unit 101 of the ultrasound apparatus 10, as shown in FIG. 5, which is a display schematic diagram of the display unit 101. In other embodiments, it is totally possible to display four images, nine images or sixteen images on the display unit at the same time, depending on how many greyscale mapping curves there are. Now, based on the images displayed on the display unit 101, the physician clicks to select the image of the best effect, for example, that image corresponding to the greyscale mapping curve A. Then, during the subsequent processing of greyscale mapping, the processing unit 102 will select the greyscale mapping relationship corresponding to the greyscale mapping curve A.

### Embodiment III

There is an application situation for the existing ultrasound apparatus, where a puncture needle is used under the guidance of an ultrasound apparatus to perform puncturing: displaying the puncture needle in real time on a display unit of the ultrasound apparatus, and a physician adjusting the puncture needle according to the image. Specifically, during puncturing, the physician needs to adjust the parameter, i.e. a needle angle of the ultrasound apparatus, such that the tip of the puncture needle is clearly displayed on the display unit. For novices or physicians who do not use this function frequently, it takes a long time to adjust this parameter to an appropriate position.

In the prior art, when the physician clicks on a button for adjusting the puncture angle, the needle angle is adjusted step by step from 10 degrees to 20 degrees, to 30 degrees till to 40 degrees where the physician finds that the tip portion of the puncture needle displayed on the screen at this moment is the most clear. However, if the adjustment is overdone, for example, the angle is adjusted to 50 degrees, the angle has to be adjusted back to 40 degrees. Now, the physician presses the button, such that the angle of the emission signal of the ultrasound apparatus is configured according to the parameter corresponding to 40 degrees. Such an operation increases the complexity of using the apparatus and also results in a waste of time.

In this embodiment, after the physician clicks the button for adjusting the needle angle (in other embodiments, it is also possible to click on a button on a touch screen), and after the ultrasound apparatus 10 receives the command, the processing unit 102 invokes pre-set values in a cache (which may be other storage devices in other embodiments, including but not limited to a floppy disk, a hard disk, an optical disk, an SD card, an internal memory, etc.). The pre-set values are 40 degrees and 80 degrees, and the two pre-set values here are empirical values widely used in the prior art. The processing unit 102 of the ultrasound apparatus 10 controls the probe 103 to emit an ultrasound pulse with a 40-degree emission angle corresponding to 40 degrees to the puncture needle and obtains an ultrasound image corresponding to 40 degrees according to an obtained ultrasound echo. Then, the processing unit 102 of the ultrasound apparatus 10 controls the probe 103 to emit an ultrasound pulse with a 80-degree emission angle corresponding to 80 degrees to the puncture needle and obtains an ultrasound image corresponding to 80 degrees according to an obtained ultrasound echo. After the processing of the ultrasound images corresponding to all the pre-set values is completed, two ultrasound images corresponding to the pre-set values are simultaneously displayed on the display unit 101. In addition, the same content as that on the display unit 101, i.e. ultrasound images corresponding to two pre-set values, is synchronously displayed on the remote display unit 104 via optical fibres, as shown in FIG. 5. Now, a remote experienced physician clicks to select an ultrasound image corresponding to 80 degrees, and then the processing unit 102 of the ultrasound apparatus 10 configures the parameter, i.e. the emission angle of the puncture needle to 80 degrees. A local physician can then carry out the next operation according to this configuration.

The present invention further provides a machine-readable storage medium, storing an instruction for enabling a machine to carry out the image processing method in an ultrasound system as described herein. Specifically, a system or device equipped with a storage medium may be provided, a software program code that implements the functions of any of the embodiments described above is stored on the storage medium, and a computer (or a CPU or an FPGA or a DSP or an MPU) of the system or device is caused to read out and execute the program code stored in the storage medium.

In this case, the program code per se read from the storage medium can realize the functions of any one of the embodiments described above, and thus the program code and the storage medium storing the program code form a part of the present invention.

The embodiments of the storage media for providing the program code include a floppy disk, a hard disk, a magneto-optical disk, an optical disk (such as a CD-ROM, a CD-R, a CD-RW, a DVD-ROM, a DVD-RAM, a DVD-RW and a DVD+RW), a tape, a non-volatile memory card and an ROM. Optionally, the program code may be downloaded from a server computer via a communication network.

In addition, it should be clear that the functions of any one of the embodiments described above can be realized not only by executing the program code read out by the computer, but also by enabling the operating system or the like operating on the computer to accomplish some or all of the actual operations based on an instruction of the program code.

In addition, it can be understood that the program code read out from the storage medium is written into a memory provided in an expansion board inserted into the computer or into a memory provided in an expansion unit connected to the computer, and then based on an instruction of the program code, a CPU or the like installed on the expansion board or the expansion unit is enabled to perform some or all of the actual operations, thereby implementing the functions of any one of the embodiments described above.

To sum up, the above is just a few illustrative examples. The pre-processing parameters and the post-processing parameters may include various parameters in various modes (including the B-mode, the colour Doppler ultrasound mode, etc.) of the existing ultrasound apparatus. For example, the pre-processing parameters include the frame frequency, focus position, frequency, depth, number of focuses, energy output, virtual convex array, packet size, etc., and the post-processing parameters include the frame average, greyscale map, pseudo-colour, suppression, lateral smoothing, etc. It should be understood that various modifications may be made to the embodiments.

This written description uses examples to disclose the invention, including the best mode, and also to enable any person skilled in the art to practice the invention, including making and using any devices or systems and performing any incorporated methods. The patentable scope of the invention is defined by the claims and may include other examples that occur to those skilled in the art. Such other examples are intended to be within the scope of the claims if they have structural elements that do not differ from the literal language of the claims or if they include equivalent structural elements with insubstantial differences from the literal language of the claims.

What is claimed is:

1. A method for configuring a parameter of an ultrasound apparatus, the method comprising:
pre-obtaining at least two corresponding ultrasound images according to at least two pre-set values of the parameter;
displaying the at least two ultrasound images simultaneously;
selecting one of the displayed ultrasound images; and
configuring the parameter to a pre-set value corresponding to the selected ultrasound image.

2. The method of claim 1, further comprising emitting an emission pulse to a detection object; and wherein pre-obtaining comprises processing a reflection pulse from the emission pulse according to the at least two pre-set values of the parameter so as to obtain the at least two corresponding ultrasound images.

3. The method of claim 1, further comprising emitting at least two emission pulses corresponding to the at least two pre-set values to a detection object, and wherein each of the ultrasound images is obtained by processing a reflection pulse from the corresponding emission pulse.

4. The method of claim 1, wherein displaying comprises displaying the at least two ultrasound images simultaneously on a remote terminal.

5. The method of claim 1, where each of the ultrasound images comprises a set of ultrasound image frames.

6. The method of claim 1, where the at least two pre-set values are empirical values of the parameter.

7. The method of claim 2, characterized in that the detection object comprises a puncture needle.

8. An ultrasound apparatus, comprising:
a processing unit, where the processing unit is configured to obtain at least two corresponding ultrasound images by means of processing according to at least two pre-set values of a parameter; and
a display unit for displaying the at least two ultrasound images simultaneously;
wherein the processing unit is further configured to receive a command of selecting one of the ultrasound images, and
where the processing unit is adapted to configure the parameter to a pre-set value corresponding to the selected ultrasound image.

9. The ultrasound apparatus according to claim 8, further comprising a probe, adapted to emit an emission pulse to a detection object, and the processing unit configured to process a reflection pulse from the emission pulse according to the at least two pre-set values of the parameter to obtain the at least two corresponding ultrasound images.

10. The ultrasound apparatus according to claim 8, further comprising a probe adapted to emit at least two emission pulses to a detection object corresponding to at least two pre-set values, where the processing unit is configured to process at least two reflection pulses corresponding to the emission pulses to obtain the at least two corresponding ultrasound images.

11. The ultrasound apparatus according to claim 8, wherein the apparatus further comprises a remote terminal configured to display the at least two ultrasound images simultaneously.

12. The ultrasound apparatus according to claim 8, where each of the ultrasound images comprises a set of ultrasound image frames.

13. The ultrasound apparatus according to claim 8, where at least two pre-set values are empirical values of the parameter.

14. The ultrasound apparatus according to claim 9, where the detection object comprises a puncture needle.
